(19) United States
(12) Patent Application Publication
(10) Pub. No.: US 2002/0187546 A1
(43) Pub. Date: Dec. 12, 2002

(54) Multi-channel packed bed bioreactor

(76) Inventors: Jacek Rozga, Westlake Village, CA (US); Achilles A. Demetriou, Bel Air, CA (US)

Correspondence Address: Edward G. Poplawski, Esq., SIDLEY AUSTIN BROWN & WOOD, 555 West Fifth Street, Los Angeles, CA, 90013-1010, US

(21) Appl. No.: 09/877,589

(22) Filed: Jun. 8, 2001

(51) Int. Cl. C12M 3/04

(52) U.S. Cl. 435/297.4; 435/299.1

(57) ABSTRACT

A bioreactor (10) supports the viability, function(s) and/or growth of a cell or enzyme, or synthesis or biotransformation (metabolism) of a composition or compound using a cell or enzyme. The bioreactor includes a housing (12) having perfusion ports (20, 24) for perfusion. A bundle of elongated elements (26) can be contained within the housing. The elongated elements can be made of a foamy polymer such that cells loaded aggregate within the pores of the elongated elements. Furthermore, the elongated elements can be a bundle of thin solid sticks (30), rod-reinforced sticks (30, 32), or thin hollow tubes (28). Optionally, the housing can include a removable cap (18, 22) configured to allow the insertion and removal of the bundle of elongated elements into and out of the housing and the bundle of elongated elements can be removably affixed within the housing.

### BACKGROUND OF THE INVENTION

1. Field of The Invention

The present invention generally to the field of biomedicine and biotechnology, and, more particularly, to an apparatus adapted for the maintenance and/or growth or propagation of cells or enzymes, or for the synthesis of a composition or compound using cells or enzymes.

2. Description of the Related Art

Various bioreactors used as cell culture devices and artificial organs are known. Typically, the known bioreactors utilize hollow-fiber technology. Various reactors, bioreactors, modules and cartridges (“BIOREACTORS”) used as cell culture devices and extracorporeal blood therapy devices [“(BIO)ARTIFICIAL ORGANS”] are known. Typically, the known bioreactors utilize hollow-fiber technology. An array of single and dual hollow-fiber reactors exists as fiulters, membrane oxygenators, plasma separators, and cell line producers and their fabrication and application are well known in the prior art, as shown, for example, by the teachings of U.S. Pat. Nos. 3,442,002; 3,492,698; 3,821,087; 3,883,393, 4,184,922; 4,219,426; 4,220,725; 4,226,378; 4,276,687; 4,283,284; 4,329,229; 4,334,993; 4,361,481; 4,374,802; 4,389,363; 4,647,539; 5,015,585; 5,605,835, 5,712,154 and other related patents.

Commonly, a bundle of small-diameter hollow, porous fibers are contained in a housing that is rigid and sealed. The bundle of fibers is stretched so that the individual fibers run in parallel to each other. The ends of the bundle are sealed at each end so that two compartments are formed: intrafiber that is within the lumens of the fibers and extrafiber that is outside the fibers but still within the housing. A biological component is loaded into the extrafiber compartment, and a perfusate is typically pumped through the intrafiber compartment. The two compartments communicate through the pores in the fiber wall. Typically, oxygen and nutrients are diffused and convected into the extrafiber space and products of cell activity move into the intrafiber space.

Conventional packed-bed bioreactor designs are also known. In one such design, a cone shaped housing contains glass beads that entrap cells, such as, liver cells. The matrix of glass beads are perfused with a perfusate.

Another packed-bed bioreactor design incorporates hollow fibers for oxygen delivery that separate layers of a polyester fabric saturated with liver cell suspension. The polyester fabric forms a roll and the perfusate, such as, culture medium or plasma, flows through the parallel channels formed by the fabric and an outer surface of the hollow fibers.

In a related design, a synthetic woven fabric is also shaped into a roll and loaded with liver cell suspension. The perfusate enters the bioreactor through a centrally positioned tube with multiple side openings, and the perfusate flows in between the layers of the cell-loaded fabric to the periphery of a cylindrical housing containing the fabric and tube.

In still another packed-bed bioreactor design, a polyurethane foam block is penetrated by a system of parallel perfusion channels, or capillaries, that run along the long axis of the block. The channels are used first to seed the foamy walls with liver cells and then to perfuse the block with a culture medium or plasma.

Although suitable for some applications, the conventional bioreactors have the some of the following drawbacks: the size is not easily tailored depending on the particular need or application; cells can not be loaded before placement in the rigid housing to take advantage of cell processing, conditioning, and culturing prior to actual use; cryopreservation and/or vitrification of cell-loaded beds and subsequent thawing and revitalization of cells prior to use is not easily achieved; uneven perfusion of seeded cells; and high cost of manufacturing.

A need therefore exists for a bioreactor that is provides solutions to the foregoing problems of the prior-art embodiments. The present invention addresses this need.

### SUMMARY OF THE INVENTION

The present invention relates to a bioreactor for growing or propagating a cell or enzyme, or for synthesizing a composition or compound using a cell or enzyme. The bioreactor includes a housing, which has a perfusion inlet port, and a perfusion outlet port. Within the housing a bundle of elongated elements made of a foamy polymer are adapted to carry cells within the pores of the elongated elements. Furthermore, the elongated elements are configured to facilitate the flow of a perfusate in between the elongated elements. The size of the bioreactor can be tailored depending on the particular need or application; cells can be loaded into the bed material before placement in the rigid housing; the cell-loaded beds can be cryopreserved and/or vitrified and subsequently thawed and revitalized prior to use; perfusion of cells can occur with minimal risk of damage to the cells or exclusion of a large number of seeded cells from contact with the perfusate; and the cost of manufacturing the bioreactor is reduced. Although the present invention is subject to a wide range of applications, it is especially suited for use in a bioreactor as a bioartificial liver, and will be particularly described in that connection.

In accordance with one aspect of the present invention, the elongated elements are a bundle of thin hollow tubes configured to facilitate the flow of the perfusate through the lumens of the tubes. Thus, the cells can be perfused from both sides of the walls of the tubes.

In accordance with another aspect of the present invention, the elongated elements are a bundle of thin solid sticks. In a more detailed aspect, a thin rigid rod is disposed within each of the thin sticks for stiffening each stick.

Other features and advantages of the present invention will be set forth in part in the description which follows and accompanying drawings, wherein the preferred embodiments of the present invention are described and shown, and in part become apparent to those skilled in the art upon examination of the following detailed description taken in conjunction with the accompanying drawings, or may be learned by practice of the present invention. The advantages of the present invention may be realized and attained by means of the instrumentalities and combinations particularly pointed out in the appended claims.

### BRIEF DESCRIPTION OF THE DRAWINGS

FIG. 1 is an exploded simplified view of a bioreactor configured according to the present invention.

FIG. 2 is a simplified view of a three elongated elements made of a foamy polymer that can be employed in the bioreactor shown in FIG. 1.

### DESCRIPTION OF THE PREFERRED EMBODIMENTS

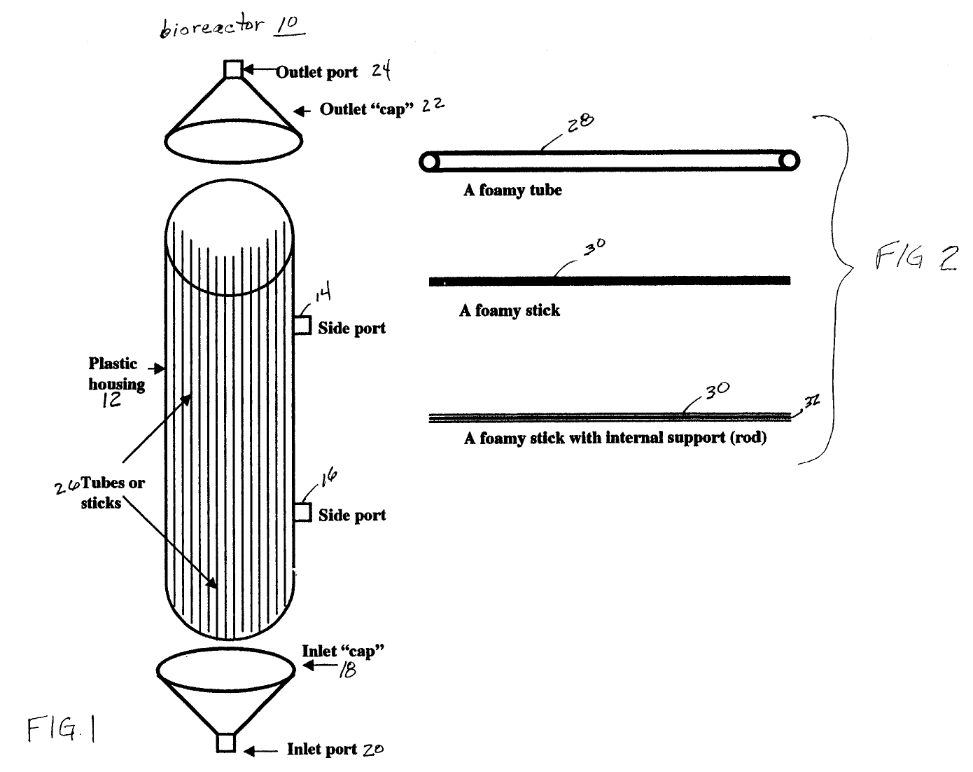

FIG. 1 illustrates a bioreactor 10 for culturing or propagating a cell or microorganism, or for synthesizing or metabolizing a composition or compound using an animal or plant cell or a microorganizm or enzyme. The bioreactor comprises a housing 12 including a perfusion inlet port 20 and a perfusion outlet port 24. Furthermore, a bundle of elongated elements 26 can be contained within the housing.

The housing 12 can be made of a rigid plastic commonly used in bioreactors and built in a conventional manner. Furthermore, in the preferred embodiment, the housing 12 can be cylindrical in shape to facilitate even perfusion of the bundle of elongated elements 26.

The elongated elements 26 can made of a foamy polymer, such as, silicone, polysulfone, or polyurethane. Cells can be loaded within the pores of the elongated elements and aggregate therein. The cells can be of any variety of interest, for example, human cells, non-human vertebrate animal, including non-human mammalian cells, insect cells, plant cells, or microorganisms, such as bacteria, fungi (e.g., yeast), or protozoans. In a preferred embodiment, the cells are mammalian hepatocytes.

Mean for conjugating functional enzymes to plastic polymers, such as silicone, polyurethane or polysulfone, are known in the art. (E.g., Drumheller, Paul D., Materials and Methods for the immobilization of bioactive species onto polymeric substrates; U.S. Pat. No. 5,914,182; Azad, A. R. M. et al., Covalent attachment of macromolecules to polysulfones or polyethersulfones modified to contain functionallizable chain ends; U.S. Pat. No. 5,462,867; Wood, et al., Preparation and use of enzymes bound to polyurethane; U.S. Pat. No. 3,928,138; Wood, et al., Use of enzymes bound to polyurethane; U.S. Pat. No. 3,929,574; Hartdegen, et al., Immobilization of proteins with polyurethane polymers; U.S. Pat. No. 4,098,645; Stormo, Keith E., Dynamic bed reactor; U.S. Pat. No. 5,531,897; Bonaventura, et al., Anti-fouling methods using enzyme coatings; U.S. Pat. No. 5,998,200; Okrongly, David, Covalent attachment of macromolecules on substrate surfaces; U.S. Pat. No. 4,933,410; Wirth, et al., Fixation of nitrogenous materials; U.S. Pat. No. 3,836,433; Axen, et al., Method of binding water-soluble proteins and water-soluble peptides to water-insoluble polymers using cyanogen halide; U.S. Pat. No. 3,645,852; Barrett, M. James, Covalently bound biological substances to plastic materials and use in radioassay; U.S. Pat. No. 4,225,784; Barker, et al., Bonding of enzymes, enzyme derivatives and other biologically active molecules to polymeric materials; U.S. Pat. No. 3,846,306; Lentfer, Dierck, Procedure for the irreversible binding of proteins onto polystyrene surfaces with retention of their biological activity, polystyrene surfaces obtained by this procedure, and their use, U.S. Pat. No. 4,654,299).

The elongated elements 26 can be fixedly disposed in the housing 12, or, optionally, the elongated elements can be modular and removably disposed in the housing. In the former version, a cell loading inlet port 14 and a cell loading outlet port 16 can be used to seed cells into the elongated elements. In the latter version, cells or enzymes can be seeded into the elongated elements outside of the housing, and the elongated elements can be disposed in the housing via a removable inlet cap 18 and/or a removable outlet cap 22.

Perfusate can flow into the perfusion inlet port 20 and out of the perfusion outlet port 24 by conventional means. The elongated elements 26 can be loosely configured in a conventional manner to facilitate the unrestricted flow of the perfusate in between the elongated elements. Thus, the cells or enzymes can remain in direct contact with the perfusate.

The dimensions of the elongated 26 can be easily tailored to meet the application of the bioreactor. For example, the elongated elements can have a length of about 10 millimeters to about 1000 centimeters, a diameter of at least about 1 millimeter, and a wall thickness of at least 20 about 0.5 millimeter. Furthermore, the size of the pores of the elongated elements can be about 200 microns to 400 microns to achieve a void volume of eighty percent.

Referring to FIG. 2, in one embodiment, the elongated elements 26 can be a bundle of thin hollow tubes 28 that are configured to facilitate not only even perfusion of the bundle of 25 elements on the outside but also the even perfusion on the inside through the lumens of the tubes.

In another embodiment, the elongated elements 26 can be a bundle of thin solid sticks 30, which, optionally, may have a thin rigid rod 32 disposed within each of the thin solid sticks 30 for stiffening each stick. The rigid rod can be made of any suitable biocompatible and/or blood compatible material, such as glass, plastic, glass, or metal, although stiff polymers, such as Teflon are preferred. A useful biocompatible and/or blood compatible material does not cause biotoxicity or other cellular injury and also does not induce a cellular or humoral immunoresponse when in contact with blood, or does not cause enzymatic inactivation.

Accordingly, because of the flexibility in the choice of the number, dimension, and placement of the elongated elements, the bioreactor can be tailored in size depending on the particular need or application, exclusion of a large number of cells from contact with the perfusate can be avoided, and the cost of manufacturing can be lowered. Furthermore, because of the portability of the elongated elements, the cells can be loaded into the bed material before placement in the rigid housing, and cell-loaded beds can be cryopreserved and/or vitrified and cells can be subsequently thawed and revitalized prior to use. Moreover, because the cells aggregate in the foamy polymer, seeded cells can be perfused with minimal risk of damage to the cells.

Those skilled in the art will recognize that other modifications and variations can be made in the bioreactor of the present present invention and in construction and operation of this bioreactor without departing from the scope or spirit of this invention.

What is claimed is:

1. A bioreactor for growing or propagating a cell or enzyme, or for synthesizing a composition or compound using a cell or enzyme, the bioreactor comprising:
a housing including,
a perfusion inlet port, and
a perfusion outlet port; and
a bundle of elongated elements made of a foamy polymer adapted to carry cells within the pores of the elongated elements, wherein the bundle of elongated elements are contained within the housing, and the elongated elements are configured to facilitate the flow of a perfusate in between the elongated elements.

2. The bioreactor of claim 1 wherein the elongated elements are a bundle of thin hollow tubes configured to facilitate the flow of the perfusate through the lumens of the tubes.

3. The bioreactor of claim 1 wherein the elongated elements are a bundle of thin solid sticks.

4. The bioreactor of claim 3 further comprising a thin rigid rod disposed within each of the thin sticks for stiffening each stick.

5. The bioreactor of claim 1 wherein the elongated elements have a length of about 10 millimeters to about 1000 centimeters.

6. The bioreactor of claim 1 wherein the elongated elements have diameter of at least about 1 millimeter.

7. The bioreactor of claim 1 wherein the elongated elements have a wall thickness of at least about 0.5 millimeter.

8. The bioreactor of claim 1 wherein the size of the pores of the elongated elements are about 200 microns to about 400 microns.

9. The bioreactor of claim 1 further comprising:
a cell loading inlet port; and
a cell loading outlet port.

10. The bioreactor of claim 1, wherein the housing further comprises a removable inlet port cap configured to allow the insertion and removal of the bundle of elongated elements into and out of the housing.

11. The bioreactor of claim 1 wherein the elongated elements are removably affixed to the housing.

12. A bioreactor for growing or propagating a cell or enzyme, or for synthesizing a composition or compound using a cell or enzyme, the bioreactor comprising:
a bundle of elongated elements made of a foamy polymer adapted to carry cells within the pores of the elongated elements; and
a housing including,
a perfusion inlet port,
a perfusion outlet port, and
a removable port cap configured to allow the insertion and removal of the bundle of elongated elements into and out of the housing;
wherein the bundle of elongated elements are removably affixed within the housing, and the elongated elements are configured to facilitate the flow of a perfusate in between the elongated elements.

13. The bioreactor of claim 12 wherein the elongated elements are a bundle of thin hollow tubes configured to facilitate the flow of the perfusate through the lumens of the tubes.

14. The bioreactor of claim 12 wherein the elongated elements are a bundle of thin solid sticks.

15. The bioreactor of claim 14 further comprising a thin rigid rod disposed within each of the thin sticks for stiffening each stick.
